SEQUENCE LISTING (22 sequences)

SEQ ID NO: 1 - DNA, 17 bp, Artificial Sequence
  Synthetic Primer
gccagcagccgcggtaa

SEQ ID NO: 2 - DNA, 21 bp, Artificial Sequence
  Synthetic Primer
cgagctgacgacarccatgca

SEQ ID NO: 3 - DNA, 29 bp, Artificial Sequence
  Synthetic Primer
ctatcaacactggagcgattctttatctg

SEQ ID NO: 4 - DNA, 30 bp, Artificial Sequence
  Synthetic Primer
ggtacttccaatggattgatgtaataaagc

SEQ ID NO: 5 - DNA, 25 bp, Artificial Sequence
  Synthetic Primer
acaacagcgctttttctaacgcttt

SEQ ID NO: 6 - DNA, 25 bp, Artificial Sequence
  Synthetic Primer
tgttagcccatattggtgctttcac

SEQ ID NO: 7 - DNA, 25 bp, Artificial Sequence
  Synthetic Primer
ggcatttaatcttgctgtatcagcg

SEQ ID NO: 8 - DNA, 25 bp, Artificial Sequence
  Synthetic Primer
tggcagcttatccgattgtacatgt

SEQ ID NO: 9 - DNA, 20 bp, Artificial Sequence
  Synthetic Primer
tgcccccgacatgtttgagt

SEQ ID NO: 10 - DNA, 24 bp, Artificial Sequence
  Synthetic Primer
attcaatccctttgtagccacacc

SEQ ID NO: 11 - DNA, 30 bp, Artificial Sequence
  Synthetic Primer
caggagctattgcaacgaaagaacaaccag

SEQ ID NO: 12 - DNA, 30 bp, Artificial Sequence
  Synthetic Primer
acatcaaaagattgaagtacatgcggatgg

SEQ ID NO: 13 - DNA, 25 bp, Artificial Sequence
  Synthetic Primer
cgggcgaaaataaaaaagaaggtaa

SEQ ID NO: 14 - DNA, 25 bp, Artificial Sequence
  Synthetic Primer
ttgccgccttatttacatgtgattt

SEQ ID NO: 15 - DNA, 20 bp, Artificial Sequence
  Synthetic Primer
tccggatccaggagcaatga

SEQ ID NO: 16 - DNA, 25 bp, Artificial Sequence
  Synthetic Primer
tccctagcaaactgctcagtacgat

SEQ ID NO: 17 - DNA, 25 bp, Artificial Sequence
  Synthetic Primer
gagaaagatgagtaaaaaacaacaa

SEQ ID NO: 18 - DNA, 21 bp, Artificial Sequence
  Synthetic Primer
catttgtgctttgaatgctag

SEQ ID NO: 19 - DNA, 21 bp, Artificial Sequence
  Synthetic Primer
ctagcattcaaagcacaaatg

SEQ ID NO: 20 - DNA, 25 bp, Artificial Sequence
  Synthetic Primer
gagaaagatgagtaaaaaacaacaa

SEQ ID NO: 21 - DNA, 244 bp, Bacillus cereus
aagcttctggtgcacaaagtgcaaacgctagttatggtacagaatttgcaactgaaacagacgtgcatgctgtgaaaaaacaaaatgctaagtcagctgcaaaacaatcacaatcttctagctcaaatcagtaatgaaagaaaaacacttctctaattcgggagaagtgttttctttttgcgtggatcgttgtaatgaaaagaaagtgaacgacataacttctaataagtcaacaaatgtagtc

SEQ ID NO: 22 - DNA, 250 bp, Bacillus anthracis
aagcttctggtgctagcattcaaagcacaaatgctagttatggtacagaatttgcaactgaaacagacgtgcatgctgtgaaaaaacaaaatgcacaatcagctgcaaaacaatcacaatcttctagttcaaatcagtaatgaaagaaaaacacttctctaactcgtgagaagtgttttctttgtgctttggccatggtaatgaaaataaatcgaacgacataacttctaataagtcaacaaatgtagtc

(19) United States
(12) Patent Application Publication
(10) Pub. No.: US 2013/0149708 A1
(43) Pub. Date: Jun. 13, 2013

(54) Multiplex PCR for Identification of B. anthracis and Detection of Plasmid Presence

(71) Applicants: Marco A. Riojas, Lorton, VA (US); Katalin Kiss, Centreville, VA (US); Marian Little McKee, Great Falls, VA (US)

(72) Inventors: Marco A. Riojas, Lorton, VA (US); Katalin Kiss, Centreville, VA (US); Marian Little McKee, Great Falls, VA (US)

(21) Appl. No.: 13/675,365

(22) Filed: Nov. 13, 2012

Related U.S. Application Data
(60) Provisional application No. 61/559,087, filed on Nov. 13, 2011.

(51) Int. Cl. C12Q 1/68

(52) U.S. Cl. CPC C12Q 1/689
USPC 435/6.12; 435/6.15

(57) ABSTRACT

The present invention includes embodiments of methods and compositions related to detection or verification of the presence or absence of Bacillus anthracis in a sample. The method embodiments include assays for the presence or absence of the pXO1 and/or pXO2 plasmids, in addition to a species-specific (such as chromosomal) marker and preferably a positive internal control.

### CROSS-REFERENCE TO RELATED APPLICATIONS

This application claims the benefit of U.S. Provisional Application 61/559,087 filed on Nov. 13, 2011, which is incorporated by reference herein in its entirety.

### STATEMENT REGARDING FEDERALLY SPONSORED RESEARCH OR DEVELOPMENT

This invention was made with United States Government support under Grant No. NIH HHSN272201000027C, awarded by the National Institutes of Health. The United States Government has certain rights in this invention.

### TECHNICAL FIELD

The field of embodiments of the present invention includes at least microbiology, cell biology, and diagnostics.

### BACKGROUND OF THE INVENTION

Bacillus anthracis is the causative agent of anthrax and is part of the closely related Bacillus cereus Group having six species including B. cereus and B. thuringiensis. B. anthracis can contain two virulence plasmids: pXO1: toxin genes (cya, lef, pag) and pXO2: capsule genes (capA, capB, capC) (see FIG. 1 for illustrations of the plasmids). Determination of whether or not a B. anthracis strain and is a biothreat and is classified as a Select Agent is based on the presence of these virulence plasmids.

The art lacks a multiplex detection process that encompasses simultaneous detection of multiple markers of significance, such as by detecting markers on both plasmids; by detecting multiple markers, including markers away from virulence genes; by detecting a chromosomal marker specific to B. anthracis; by simultaneously determining plasmid presence and species-level identification; by using an internal positive control; and by using one or more targets that will reliably amplify.

### BRIEF SUMMARY OF THE INVENTION

The present invention is directed to systems, methods, and/or compositions related to detection, identification, or verification of Bacillus anthracis. In some embodiments, there is molecular authentication of Bacillus anthracis at the species level and/or detection and/or characterization of its virulence plasmids. In some embodiments, the methods identify an organism in question as being B. anthracis, whereas in some embodiments the methods of the invention identify that an organism is not B. anthracis. In some embodiments, an organism is suspected of being B. anthracis and the methods of the invention are employed to verify such; the verification may confirm or refute that the organism is B. anthracis. A sample comprising an organism that is in need of being tested as being B. anthracis or verified as being B. anthracis may be the source of the entity or entities being tested. The sample may be provided to the party performing the inventive method, or the party performing the inventive method may directly obtain the sample. The sample to be tested may be obtained directly or indirectly from a repository of bacterial organisms and in need of verification, or the sample to be tested may be provided to a repository of bacterial organisms and in need of verification.

Embodiments of the assay are useful to characterize a sample that is or that is suspected of comprising B. anthracis. In specific embodiments, genomic DNA of the organism in question is employed to simultaneously provide an identification of Bacillus anthracis, as well as to determine which, if any, of the virulence plasmids are present. In certain embodiments of the invention, there is a detection assay of unknown genetic or bacterial material to determine if a sample is or contains Bacillus anthracis (or to rule it out through negative results); in at least some cases one determines whether the virulence plasmids are present.

In some embodiments of the invention, purified genetic material of the sample in question is utilized in detection methods. In certain embodiments of the invention, bacterial material is employed in detection methods. Certain aspects of the methods will allow determination of Select Agent status of a bacterial sample. In some embodiments of the invention, an unknown substance (such as a powder or liquid, for example) is subjected to method(s) of the invention, for example to determine if it comprises hazardous material. In some embodiments, the method is utilized to identify or verify a sample suspected of being B. anthracis. In certain cases, one can utilize the invention in biodefense embodiments, such as to test for a substance suspected of being used in biological warfare or poses a threat by being suitable for use in biological warfare.

In particular embodiments, an assay is provided that detects not only the presence or absence of pXO1 and/or pXO2 in a sample suspected of comprising B. anthracis but that also utilizes species-specific analysis. In some embodiments of the invention, a method is employed to determine the presence of one or both of pXO1 and pXO2 plasmids in an organism, irrespective of whether or not the organism is known or suspected to be B. anthracis. In some embodiments of the invention, a method is employed to determine the presence of plasmids with regions of sequence homology to one or both of pXO1 and pXO2 plasmids in an organism, irrespective of whether or not the organism is known or suspected to be B. anthracis.

In some embodiments, there is performing of one or more multiplex PCR amplifications for the purpose of simultaneously identifying Bacillus anthracis and/or detecting pXO1 and pXO2 plasmid presence in Bacillus anthracis strains.

Embodiments of the invention encompass a novel multiple-target multiplex PCR assay capable of simultaneous species level identification of Bacillus anthracis via a unique chromosomal marker and/or the detection of the pXO1 and pXO2 plasmids via multiply redundant targets on each. The assay is useful to characterize a known or unknown sample containing or potentially containing Bacillus anthracis bacteria or bacterial genomic DNA by simultaneously 1) detecting whether the tested sample contains DNA sequences specific to Bacillus anthracis, and 2) determining which, if any, of the virulence plasmids common to Bacillus anthracis (pXO1 and pXO2) are present. In certain aspects, there are multiple targets in the invention including 2, 3, 4, 5, 6, 7, 8, 9, 10, 11, 12, or more targets.

In particular aspects of the invention, there is a multiplexed combination of primers that target: a) a chromosomal mutation specific to Bacillus anthracis (sspE repeat), allowing species-level identification of B. anthracis; b) four multiply redundant targets (the three toxin genes, lef, pag, and cya, and a separate target located distant from the pXO1 pathogenicity island, ORF53) on the pXO1 virulence plasmid, allowing detection and characterization of the pXO1 virulence plasmid; c) three multiply redundant targets (two capsule genes, capA and capB, and a separate target located distant from the location of the pXO2 capsule genes, ORF7) on the pXO2 virulence plasmid, allowing detection and characterization of the pXO2 virulence plasmid; and d) the 16S ribosomal RNA (rRNA), allowing the target to function as an internal control that should be amplified in nearly all bacterial genomic samples.

In some embodiments of the invention, the methods are employed for verification of insertions, mutations, and/or deletions of targeted regions. In some embodiments the invention is useful as a verification assay of altered biological properties, such as insertions, mutations, inversions, deletions, and so forth. For example, those performing deletions of regions targeted by embodiments of this assay could use the absence of a band in the results to verify the successful deletion of one or more regions, in certain embodiments. In some cases, those performing insertions or mutations in one or more regions targeted by this assay could use the difference in band size to verify the successful insertion into or mutation of the region.

In some embodiments, there is a method of testing for the presence or absence of Bacillus anthracis in a sample, comprising the steps of assaying for the presence of the following targets in nucleic acid from the sample: a species-specific target; two or more targets on pXO1 plasmid, wherein a first target is a virulence gene and a second target is distant on the plasmid from the first target and/or is a non-virulence gene; two or more targets on pXO2 plasmid, wherein a first target is a virulence gene and a second target is distant on the plasmid from the first target and/or is a non-virulence gene; and optionally a bacterial genomic positive control target. In specific embodiments, the method is further defined as assaying for two or three virulence gene targets on pXO1 plasmid. In certain embodiments, the method is further defined as assaying for two or three virulence gene targets on pXO2 plasmid. In specific aspects, the species-specific target comprises sspE (including a mutation in sspE) or is one or more B. anthracis-specific prophage(s).

In specific aspects, first target on the pXO1 plasmid is selected from the group consisting of lef, pag, cya, and combinations thereof. In certain aspects, a first target on the pXO2 plasmid is selected from the group consisting of capA, capB, capC, capD, capE, and combinations thereof. In certain embodiments, a second target on the pXO1 plasmid is ORF53. In some embodiments, a second target on the pXO2 plasmid is ORF7.

In specific embodiments, bacterial genomic positive control target is a housekeeping gene, such as ribosomal RNA, including 16S rRNA.

In some cases the method includes the step of obtaining the sample.

In some embodiments, a sample is suspected of comprising B. anthracis or known to comprise B. anthracis. In some cases, a sample is or is from a powder, liquid, gel, aerosol, solid, or mixture thereof. The sample may be from or is an unknown substance. In certain aspects, a sample is from a repository and/or is to be deposited in a repository. In certain cases, the method further comprises the step of transporting the sample.

In specific embodiments, the nucleic acid is purified nucleic acid. In some embodiments, the assaying comprises amplification of one or more of the targets, such as by polymerase chain reaction. In specific embodiments, the method comprises the steps of assaying for the presence of the following targets in nucleic acid from the sample: sspE; lef, pag, and cya; ORF53; ORF7; capA and capB; and 16S RNA.

In some embodiments, there is a kit comprising primers suitable for amplification of the following targets: a species-specific target; one or more targets on pXO1 plasmid, wherein a first target is a virulence gene and a second target is distant on the plasmid from the first target and/or is a non-virulence gene; one or more targets on pXO2 plasmid, wherein a first target is a virulence gene and a second target is distant on the plasmid from the first target and/or is a non-virulence gene; and optionally a bacterial genomic positive control target.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 illustrates exemplary targeted regions on the pXO1 and pXO2 plasmids;

FIG. 2 provides an exemplary amplicon of sspE for B. anthracis;

FIGS. 3A-3B show exemplary assay results. A) A graphic representation of the gel electrophoresis results from the multiplex PCR of a pXO 1⁺/pXO2⁺Bacillus anthracis sample, e.g. NR-411. B) Gel electrophoresis results from the multiplex PCR of DNA from NR-411 (pXO1⁺/pXO2⁺) and NR-1400 (pXO1⁺/pXO2⁻);

FIGS. 4-7 show exemplary inclusivity/exclusivity results. Samples of B. anthracis as well as from other genera and kingdoms are listed on the leftmost column of the figures. The results are coded: dark gray indicates that an amplicon for the particular target was observed via gel electrophoresis, while light gray indicates that no amplicon was observed for a particular target.

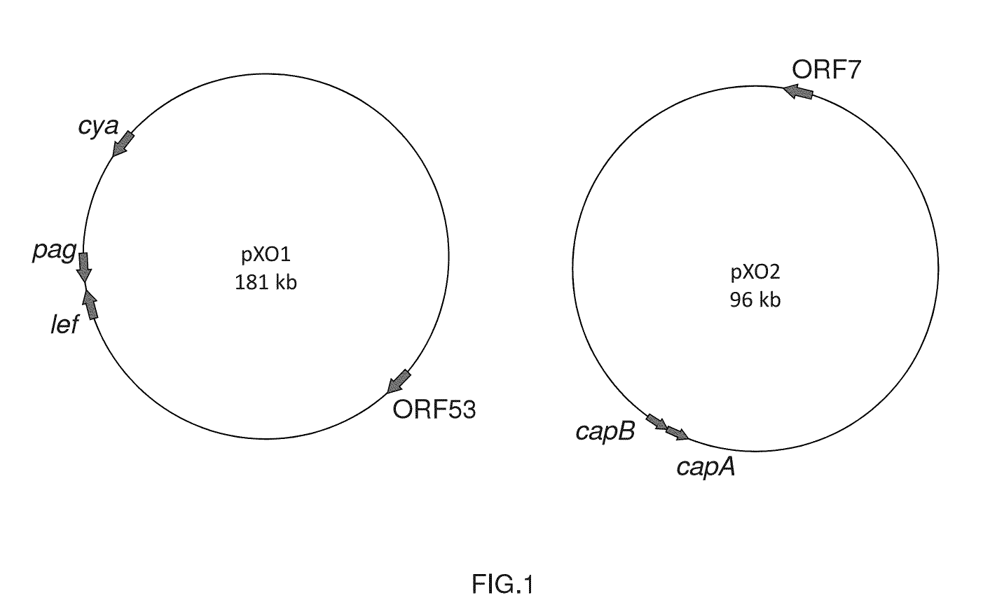

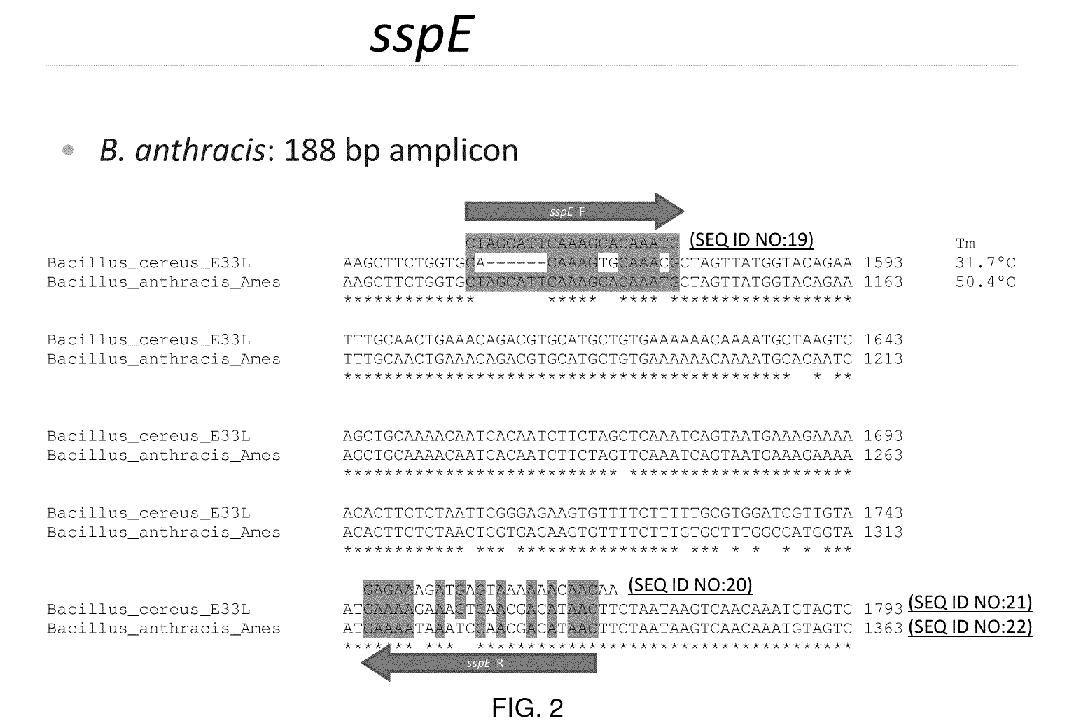

### DETAILED DESCRIPTION OF THE INVENTION

### I. Exemplary Definitions

16S rRNA: A component of the ribosomal ribonucleic acid (rRNA) common to all prokaryotes. Although the sequence of the 16S region can vary between species and strains, universal primers can generally be used to amplify the 16S region of rRNA.

Amplicon: The product of a PCR reaction, resulting from the repeated amplification of a specific region of DNA.

Genomic DNA region: A large fragment of DNA that is approximately 1,000 to 3,000 base pairs (bp) long, although in some cases the fragment is shorter or longer.

Multiplex PCR: A variant of PCR in which a single reaction mixture containing various primer pairs is used to amplify multiple regions of genomic and/or plasmid DNA simultaneously.

PCR Mastermix: A mix of reagents that includes all components necessary for PCR (e.g., DNA polymerase, dNTPs, MgCl₂, etc.) except the DNA templates.

Polymerase chain reaction (PCR): A technique for amplifying DNA sequences in vitro by separating the DNA into 2 strands and incubating them with primers, a thermostable DNA polymerase, and other various necessary reaction components. PCR can amplify a specific sequence of DNA greater than a million-fold.

Primer: A segment of DNA, around 20 to 40 bases, that is complementary to a given nucleic acid sequence and is needed to initiate replication by DNA polymerase.

Primer dimer: Two segments of primers anneal to each other rather than the given complementary DNA sequence.

Primer mix formulation: A solution containing all the primer sets to be used in multiplex PCR, at their appropriate relative concentrations.

Reagent blank: A sample that is run with only reagents (no template), in parallel with the DNA extraction procedure; it is used in a PCR reaction to verify the absence of a DNA contaminant in the extraction reagents.

Template DNA: A single strand of DNA that will be used to create a complementary strand of DNA. The complementary bases will form bonds between the original strand of DNA and the complementary strand of DNA.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

### II. General Embodiments of the Invention

In embodiments of the invention, there are methods and compositions that regard assaying for the presence or absence of B. anthracis, wherein such assaying employs assaying for at least one plasmid in B. anthracis, at least one chromosomal target in B. anthracis, and at least one control for detection of a bacterial species. In specific embodiments, one or more targets are the subject of one or more assays for detection of at least one plasmid from B. anthracis. In specific embodiments, at least two targets per plasmid are included in an assay as an object of identification of B. anthracis. In particular cases, at least one of the targets on the plasmid is a virulence gene, whereas at least one of another of the targets on the plasmid is not a virulence gene.

General embodiments of the invention encompass multiplexed detection and characterization of B. anthracis, and in specific embodiments it concerns detection and characterization of B. anthracis vs. an organism that is not B. anthracis. In specific aspects, the methods concern identification of four targets on pXO1, including 3 toxin genes and 1 control gene; three targets on pXO2, including 2 capsule genes and 1 control gene; and at least one internal positive control (16S rRNA, for example). In particular embodiments, there is rapid characterization of a sample, for example, within the order of a few hours. A low limit of detection for extracted DNA, such as ˜30 pg, is possible. In specific embodiments, a representative sample of a sample in question is assayed for the particular bacteria, and the assaying may be performed on or from dead or live bacteria.

In specific embodiment, the invention encompasses a novel multi-target multiplex PCR assay capable of simultaneous species-level identification of Bacillus anthracis via a unique chromosomal marker and the detection of the pXO1 and pXO2 plasmids via multiply redundant targets on each. In certain embodiment, the assay is useful to characterize a known or unknown sample containing or potentially containing Bacillus anthracis bacteria or bacterial genomic DNA by simultaneously 1) detecting whether the tested sample contains DNA sequences specific to Bacillus anthracis, and 2) determining which, if any, of the virulence plasmids common to Bacillus anthracis (pXO1 and pXO2) are present.

A. Exemplary Uses of the Invention

The invention is useful as a detection assay of purified genetic material, bacterial material, or unknown substances (such as powders, potentially hazardous material, etc.) to determine if the sample is or contains Bacillus anthracis (or to rule it out through negative results) and whether the virulence plasmids are present. Other embodiments include those for verification and/or characterization of Bacillus samples, particularly with regard to positively or negatively identifying a Bacillus sample as a Select Agent. Other embodiments include those for research purposes, such as to verify whether or not certain genetic alterations have been made or are otherwise present, including insertions, mutations, or deletions of targeted regions. A skilled artisan performing one or more deletions of one or more regions targeted by embodiments of the invention may, for example, use the absence of a band in the results to verify the successful deletion of the region. Analogously, skilled artisans performing insertions or mutations in the regions targeted by this assay could ascertain the difference in band size to verify the successful insertion into or mutation of the region.

The material to be tested may be a powder, liquid, gel, from a swab or stab or any unknown substance. The source of bacteria/DNA could also include an aerosol, though in some embodiments such testing generally deposits aerosolized particulates onto a solid surface (typically a filter) or into a liquid medium.

In specific embodiments, suspicious items or locations/environments suspected of being contaminated could be swabbed and tested from the solid swab surface, or the particulates from the swab could similarly be re-suspended in a liquid medium, for example.

B. Targets for Multiplex PCR for Identification of B. anthracis

In embodiments of the assay, there is a multiplexed combination of primers that target: a) a chromosomal location specific to Bacillus anthracis, allowing species-level identification of B. anthracis, b) two or more multiply redundant targets, at least one of which is a separate target located distant from the pXO1 pathogenicity island on the pXO1 virulence plasmid, allowing detection and characterization of the pXO1 virulence plasmid, c) two or more multiply redundant targets at least one of which is a separate target located distant from the location of the pXO2 capsule genes on the pXO2 virulence plasmid, allowing detection and characterization of the pXO2 virulence plasmid, and d) a target that functions as an internal control that should be amplified in nearly all bacterial genomic samples.

In embodiments of the assay, there is a multiplexed combination of primers that target: a) a chromosomal mutation specific to Bacillus anthracis (sspE repeat), allowing species-level identification of B. anthracis, b) four multiply redundant targets (the three toxin genes, lef, pag, and cya, and a separate target located distant from the pXO1 pathogenicity island, ORF53, for example) on the pXO1 virulence plasmid, allowing detection and characterization of the pXO1 virulence plasmid, c) three multiply redundant targets (two capsule genes, capA and capB, and a separate target located distant from the location of the pXO2 capsule genes, ORF7, for example) on the pXO2 virulence plasmid, allowing detection and characterization of the pXO2 virulence plasmid, and d) the 16S ribosomal RNA (rRNA), allowing the target to function as an internal control that should be amplified in nearly all bacterial genomic samples.

1. Plasmid Targets

In embodiments of the invention, a plasmid in B. anthracis is detected as part of identification of B. anthracis. In specific embodiments, the presence of both of pXO1 and pXO2 plasmids is assayed, although in alternative embodiments only one of the plasmids is the subject of the assay. In particular embodiments, at least two targets on a plasmid are included in the multiplex analysis. In certain embodiments, one of the targets in the analysis is a non-virulence gene and/or a gene that is distant from a particular locus, such as the pXO1 pathogenicity island and the pXO2 capsule gene(s) (see Okinaka et al. and Vander Auwera et al., 2005, for example). In specific embodiments, ORF53 on pXO1 and ORF7 on pXO2 are utilized as a locus distant from the pXO1 pathogenicity island and the pXO2 capsule gene(s), respectively.

In specific embodiments, the presence of at least one virulence gene on pXO1 and pXO2 is included in the assay. In specific cases, one or more of lef, pag, and cya are included in the assay for pXO1. In specific cases, one or more of capA, capB, capC, capD, and capE are included in the assay for pXO2. Another virulence gene that may be employed is atxA, which is a global virulence regulator located on pXO1.

Such embodiments of the invention allow for detection of the respective plasmid presence in the event of the mutation or deletion of the main virulence targets (lef, pag, and cya on pXO1, and capA, capB, and capC on pXO2, for example). Because these targets are the main plasmid virulence genes in B. anthracis, they are the most likely to be intentionally mutated or deleted in a research strain, for example. Mutating, deleting, or otherwise rendering unamplifiable all of these targets on a given plasmid would prevent many assays from detecting them, resulting in false negative results. The inclusion in the current invention of additional targets located distant from the main genes of interest was designed to circumvent this flaw and provide for appropriate detection in such strains.

2. Chromosomal Target(s)

Because plasmids such as pXO1 and pXO2 have the capability of horizontal transfer to other bacterial species, their presence is not necessarily indicative of Bacillus anthracis. Detection of at least one chromosomal marker unique to B. anthracis is useful to make a correct species characterization of a bacterium as B. anthracis. Few assays are designed to use a chromosomal target to identify Bacillus anthracis to the species level. Most of those that do use a target that has since been shown to not be specific to B. anthracis (Ba813). The Ba813 marker amplified these assays was initially described as being specific to B. anthracis (Patra, et al., FEMS Immunol Med Microbiol, 1996, 223). It was later shown to exist in certain strains of B. cereus and B. thuringiensis (Ramisse, et al., J Appl Microbiol, 1999, 224). Although the data from Ramisse, et al. show detection in all B. anthracis strains tested, its presence in two other species indicates that the sequence is not restricted to B. anthracis. Although Ba813 may necessarily be present in B. anthracis, presence of the sequence does not necessarily identify a sample as B. anthracis. Thus, Ba813 is insufficiently specific to use as a marker for B. anthracis.

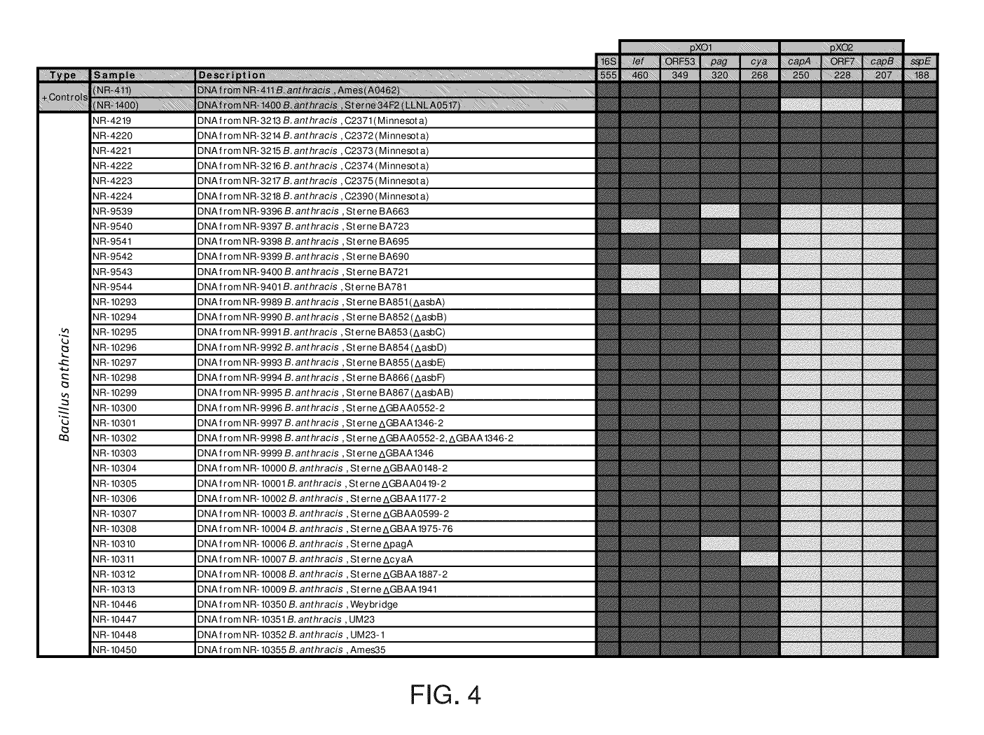

In embodiments of the invention, the unique chromosomal mutation in the sspE gene targeted by the current invention has not been identified outside of B. anthracis; therefore, it is a better specific indicator of B. anthracis (see FIG. 4).

The sspE primer pair from Kim, et al. is designed to produce a 75 bp amplicon in species from the B. cereus group of organisms (B. cereus, anthracis, thuringiensis, mycoides, etc.) In B. anthracis, these primers also produce a 188 bp amplicon due to a species-specific repeated region that includes the binding site for the reverse primer.

In alternative embodiments, a chromosomal target specific for B. anthracis other than sspE is employed in the assay, such as B. anthracis-specific prophage (see, for example, Sozhamannan et al., 2006). Any primer or primers may be used for detection of sspE so long as they produce a PCR product that is specific to the sspE region being amplified and allows accurate identification of the presence of sspE.

3. Positive Control

In embodiments of the invention, a control that identifies a sample as having bacteria is employed in the invention. The control may be considered to be an internal control for bacterial genomic DNA, in certain embodiments. The inclusion of a 16S rRNA target amplified by universal primers (for example) that should bind to sequences from nearly all bacteria allow the amplicon to function as a native internal control, showing the success of the amplification reaction regardless of the other results obtained (e.g., not B. anthracis, pXO1⁻, and/or pXO2⁻.)

### III. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or plasmid DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the respective targets are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCRTM) which is described in detail in U.S. Pat. Nos. 4,683,195, 4,683,202 and 4,800,159, and in Innis et al., 1988, each of which is incorporated herein by reference in their entirety.

A reverse transcriptase PCR amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCRTM and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### IV. Detection of Nucleic Acids

Following any amplification, it may be desirable to separate amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

### V. Kits of the Invention

Any of the compositions described herein may be comprised in a kit. In a non-limiting example, one or more primers and/or reagents for one or more targets of the invention may be comprised in a kit, wherein the primers are in suitable container means. The components of the kits may be packaged either in aqueous media or in lyophilized form. The container means of the kits may generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the primers and any other reagent containers in close confinement for commercial sale. Such containers may include injection or blow-molded plastic containers into which the desired vials are retained.

When the components of the kit are provided in one or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

In specific embodiments of the invention, primers suitable for amplifying one or more targets of the invention are utilized in the kit. Such primers may allow amplification of all or part of a locus in question. Primers may allow amplification of sspE, including a mutation in sspE; lef; pag; cya; ORF53; ORF7; capA; capB; capC; and/or 16S RNA.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials, Supplies and Reagents

All items, including disposable items, are preferably sterile.

A. Exemplary Materials and Supplies
1. PCR tubes (VWR® 93001-118 or equivalent)
2. Microcentrifuge tubes for making mastermix [e.g., 1.5 mL tubes (VWR® #20170-038 or equivalent)]
3. Appropriate size tube racks
4. 4% agarose E-Gel® [Invitrogen™ #G5018-04 (contains ethidium bromide, see step VI.A)] or equivalent
5. Appropriate DNA ladder (Invitrogen™ #10488-043 or equivalent)
6. Appropriate micropipettes
7. Appropriate RNase-free and DNase-free aerosol-resistant micropipette tips
8. Appropriate disposable gloves
9. Bench-top cooler (ice block or equivalent)

B. Exemplary Reagents
1. Molecular Biology Grade Water [ATCC® #60-2450 or equivalent molecular biology grade (MBG) water]
2. Platinum® Taq DNA Polymerase Kit (Invitrogen™ #10966-034), kit includes the following:
   a. Platinum® Taq DNA polymerase 5 U/rxn
   b. Magnesium chloride (MgCl₂), 50 mM
   10×PCR Buffer
3. 2.5 mM dNTP mix (2.5 mM each dNTP)
4. Sterile PCR H₂O
5. Primers
   a. Appropriate primers (see Table 1—Primer Sequences and Expected Amplicon Sizes)
6.70% ethanol

C. Exemplary Standards and Controls
1. Positive Control (diluted to a concentration of 2 ng/μL, as necessary)
   a. DNA from BEI# NR-411 (or equivalent pXO1⁺/pX02⁺B. anthracis strain)
   b. DNA from BEI# NR-1400 (or equivalent pXO1⁺/pXO2⁻B. anthracis strain)
2. Bacterial Negative Control
   a. BEI DNA# NR-2541 (or equivalent pXO1⁻/pXO2⁻ non-B. anthracis strain)
3. Reaction Negative Controls
   a. A reagent blank can be used as a clean extraction control.
   b. MBG is used as a no-template reaction control.

D. Samples
1. Template DNA (diluted to a concentration of 2 ng/μL, as necessary)

### EXEMPLARY EQUIPMENT

A Biological safety cabinet (BSC) or PCR hood

B. Microcentrifuge

C. Vortexer

D. Programmable thermocycler

E. Electrophoresis chamber and power supply

F. Gel photodocumentation device (e.g., Bio-Rad Gel Doc™ XR or equivalent)

### SAFETY PRECAUTIONS

A. Caution: Ethidium bromide is a mutagen and may alter genetic material. Avoid inhalation, contact with eyes, skin and clothing, as well as, prolonged or repeated exposure. Wash hands thoroughly after handling.

B. Use appropriate personal protective equipment and laboratory containment when executing methods of the invention.

C. Do not use reagents past the expiration date or suggested shelf life.

D. Before use, check that the packaging of the various components is intact. Do not use any components that have been damaged.

### LIMITATIONS AND POTENTIAL INTERFERENCES

A. All solutions used in the methods are preferably free of contaminating nucleases and nucleic acids. To minimize the risk of contamination, use clean gloves while handling and preparing materials and solutions. If possible, all solutions should be prepared in sterile and nuclease-free containers, using nuclease-free and nucleic acid-free micropipette tips.

B. Proper care of template DNA should be observed. In particular, repeated freeze-thaw cycles should be avoided. Large volume stocks of template should be divided into smaller volume aliquots, and only 1 of these vials should be thawed at a time. Vial of template DNA should be thawed at 4° C. Template DNA should NEVER be vortexed, as the long strands of genomic DNA and plasmids are extremely vulnerable to shearing in such conditions. Template DNA is properly re-suspended by flicking the tube multiple times.

C. Make sure that the biological work area where the PCR setup will be performed is cleaned properly with 70% ethanol.

### PREREQUISITES

A Utilize target-specific primers (see Table 1—Primer Sequences and Expected Amplicon Sizes) for the targets of interest.
1. Lyophylized primers can be stored at room temperature.
2. Once rehydrated, primers should be aliquoted and stored at approximately −20° C. for long term storage. Liquid primers may be kept at 2 to 8° C. for a week while in use.

B. Preparation of Primer Stock Solutions
1. Stock Solution of Primers
   a. Calculate the volume of MBG water to add to each primer vial in order to achieve a 100 μM stock solution.
   b. Add the appropriate amount of MBG water to each primer vial.
   c. Vortex briefly at high speed.
   d. Microcentrifuge at maximum speed for 2 to 5 seconds, to remove solution from the sides of the tube.
   e. Store at −20° C. or colder. If making 20 μM working stock solutions, immediately make dilutions from the stock solutions, then store the stock solutions at −20° C. or colder.
2. Preparation of 50 μM Primer Working Stock Solution of lef Primers
   a. In a labeled microcentrifuge tube, add 20 μL of MBG water with 20 μL of the 100 μM stock solution primer.
Note: If a large number of reactions are being run, the volume necessary of some primers may be greater than 40 μL If this is the case, instead create a working stock solution with a sufficient final volume, making sure that the concentration remains 50 μM (i.e., 1 part stock primer solution, plus 1 part MBG).
   b. Vortex briefly at high speed to mix solution together.
   c. Microcentrifuge at maximum speed for 2 to 5 seconds, to remove solution from the sides of the tube.
   d. If not using immediately, store at −20° C. or colder.
3. Preparation of 20 μM Primer Working Stock Solutions (all except lef primers)
   a. In a labeled microcentrifuge tube, add 32 μL of MBG water with 8 μL of the 100 μM stock solution primer.
Note: If a large number of reactions are being run, the volume necessary of some primers may be greater than 40 μL If this is the case, instead create a working stock solution with a sufficient final volume, making sure that the concentration remains 20 μM (i.e., 1 part stock primer solution, plus 4 parts MBG).
   b. Vortex briefly at high speed to mix solution together.
   c. Microcentrifuge at maximum speed for 2 to 5 seconds, to remove solution from the sides of the tube.
   d. If not using immediately, store at −20° C. or colder.

C. Preparation of Primer Mix Formulation
1. To a new labeled microcentrifuge tube, add the volume specified in the shaded column of Table 2 (see exemplary Table 2) from each of the 20 μL primer working stocks.
Note: This primer mix contains enough volume for 10 reactions. If more than 10 reactions are being performed, the volume of each primer added to the primer mix can be increased (e.g., doubled, tripled, etc.) in order to ensure sufficient primer mix.
2. Vortex briefly at high speed to mix solution together.

D. If one does not already exist, create a 20 μL working stock of each template DNA (including controls) at a concentration of 2 ng/μL.
1. Perform the following calculation:

(Concentration of DNA ng/μL)(Volume to add X μL)=(2 ng/μL)(20 μL)

Example: DNA concentration of 25 ng/μL

(25 ng/μL)(X μL)=(2 ng/μL)(20 μL) X=1.6 μL DNA to add to make working stock
2. Add the DNA volume calculated above (X) to a fresh, labeled microcentrifuge tube.
3. Add a volume of MBG sufficient to make the total volume 20 μL.

Example: DNA concentration of 25 ng/μL

Added 1.6 μL DNA: 20 μL-1.6 μL DNA=18.4 μL MBG H₂O

Note: If the volume of DNA sample to add is very small (i.e., <1 μL), prepare a 100 μL working stock of template DNA instead.

### Example 2

### Exemplary Procedure Preparation

A. Overview of the Test Procedure.

In general, a biological work area is prepared and, if necessary, primers and template DNA are prepared; a mastermix is prepared and loaded into PCR tubes. Then, in a BSC, DNA is loaded into PCR tubes. An appropriate thermocycler protocol is run for PCR. The PCR products are run on an agarose gel followed by picturing of the gel and analysis of results and data.

B. General Information
1. Completing one step may be dependent on information given in a later step. Prior understanding of sequential steps is critical to the success of this procedure.
2. Always wear gloves while handling reagents and nucleic acid samples to prevent nucleic acid contamination from the surface of the skin or from laboratory equipment. Hands and dust particles may carry bacteria and molds and are the most common sources of contamination. Change gloves frequently (especially if contact between gloves and sample occurs) and keep tubes closed whenever possible.
3. When setting up PCR reactions, make sure to add the template DNA to the PCR reactions last. Mix and add all other reagents first.

Put the containers away. Then bring out the template DNA and add it to each PCR reaction.

### Example 3

### Exemplary Procedure of the Invention

1. Preparation
a. Thaw the following:
   1) Platinum® Tag DNA Polymerase 5 U/rxn
   2) Magnesium Chloride (MgCl₂), 50 mM
   3) 10×PCR Buffer
   4) 2.5 mM dNTP Mix (2.5 mM each dNTP)
   5) Sterile PCR H₂O
   6) Primers (see Table 1—Primer Sequences and Expected Amplicon Sizes)
   7) DNA Templates (2 ng/μL), both controls and unknowns
b. Label all tubes with a permanent marker

2. Setup
a. Prepare mastermix according to Table 2. Note: Keep Platinum® Taq DNA Polymerase in bench-top cooler until ready for use. Once Taq is added to the mastermix, add mastermix as quickly as possible to each PCR reaction.
b. Add to each labeled PCR tube:
   1) 46 μL of mastermix
   2) 4 μL of template DNA at a concentration of 2 ng/μL.

Add the template in a template designated area (e.g., BSC or PCR hood).
c. Spin samples down in a microcentrifuge, for 2 to 5 seconds, at maximum speed.

3. PCR

Using a thermocycler, perform PCR of samples using the following protocol (exemplary only):

| Step | Temp | Time | | # of Cycles |
|---|---|---|---|---|
| Pre-heat | 95° C. | 5 | min | 1x |
| Denature | 95° C. | 30 | sec | 35X |
| Anneal | 62° C. | 30 | sec | |
| Extend | 72° C. | 1 | min | |
| Final Extension | 72° C. | 2 | min | 1x |

4. Run Gel
a. Run PCR product on an agarose gel
   1) The recommended conditions are:
      a) Use a 4% agarose E-Gel® (Invitrogen™)
      b) Load a mixture of 10 μL PCR product and 10 μL 1:25 10× BlueJuice (Invitrogen™)
      c) Load 10 to 15 μL 50 bp DNA ladder (Invitrogen™)
      d) Run the gel for one 30-minute run, followed immediately by one 15-minute run.
   2) Record equipment used and product lot information.

5. Photodocumentation
a. Document the results with a photodocumentation device.

### EVALUATION AND INTERPRETATION OF RESULTS

Calculations are performed algorithmically.

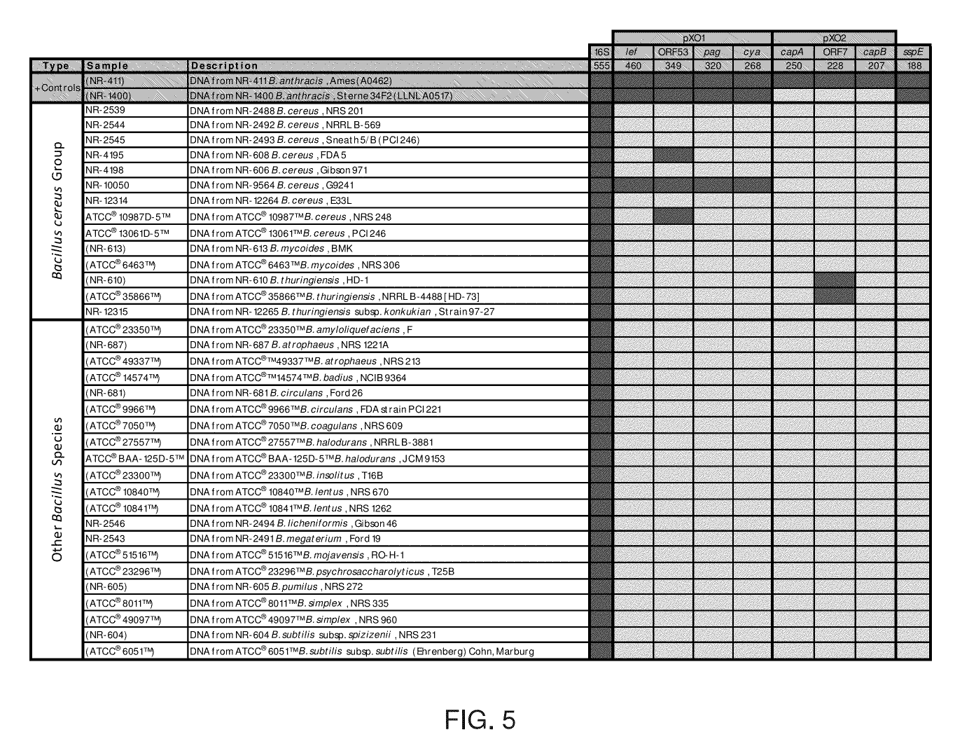

A. If the appropriate template DNA is present in the sample, each PCR primer pair will generate an amplicon(s) that can be visualized as a band(s) at a specific size(s) on the agarose gel. These bands are evident once a picture of the gel is taken with a gel photodocumentation system. If a band in the PCR product is the same as the expected product size (see FIG. 5 for expected product sizes), then the PCR was successful in identifying the corresponding gene target. The size of the bands may vary slightly due to slight differences between individual strains, but should be generally consistent with the representative results.

B. Satisfactory Test Evaluation
1. The PCR reaction is satisfactory (passes) if all of the following conditions are met:
   a. The reaction negative control shows no bands.
   b. The bacterial negative control shows no bands, other than a 16S band (as an example), consistent with the size of the expected product(s).
   c. The PCR products of the bacterial positive controls are consistent with the size of the expected product(s).
   d. The test sample(s) contain only band(s) of a size consistent with the size of the expected product(s).
2. The PCR reaction is unsatisfactory (fails) if any of the following conditions are met:
   a. The reaction negative control shows a band.
   b. The bacterial negative control shows a band, other than a 16S band (as an example), consistent with the size of the expected product(s).
   c. The PCR products of the bacterial positive controls do not contain bands consistent with the size of the expected product(s).
3. Individual test samples are unsatisfactory (fail) if any of the following conditions are met:
   a. The test sample(s) show a band of an unexpected product size.
   b. The test sample(s) show no bands consistent with the size of the expected 16S product and no band consistent with the size of the other expected product(s).
4. Results

In exemplary embodiments, the 16S band provides an “internal positive control.” Because the 16S gene is present in all bacteria, nearly all samples tested with the universal primers in this multiplex PCR will display a 16S band. Thus, this band is used to indicate both the presence of sample DNA and also the proper functioning of the PCR reaction. The absence of a 16S band coupled with the absence of other bands suggests an unsuccessful PCR.

C. Characterization of Test Samples
1. Species Characterization
   a. If the PCR product shows a band at approximately 188 bp, the sample is consistent with Bacillus anthracis.
   b. If the PCR product does not show a band at approximately 188 bp, the sample is not consistent with Bacillus anthracis. (Alternatively, a B. anthracis sample with a mutation/deletion of this sspE gene target could present with similar results.)
2. Plasmid Characterization
   a. pXO1
      1) If all of the bands at approximately 460, 349, 320, and 268 bp are present, the sample contains the pXO1 plasmid or a plasmid with DNA sequence homology in the regions of the particular targets (e.g., pBCXO1 in NR-9564 B. cereus) and should be characterized as pXO1⁺.
      2) If some (but not all) of the bands at approximately 460, 349, 320, or 268 bp are present, the species of the sample should be considered.
   a) If the sample is DNA from B. anthracis, it is likely that it contains the pXO1 plasmid, possibly with a mutation/deletion for one or more of the targets, and should be characterized as pXO1+.
   b) If the sample is not DNA from B. anthracis and not from a strain known to harbor a homologous plasmid (e.g., pBC10987 in ATCC® 10987™B. cereus), the strain may contain a plasmid with DNA sequence homology to pXO1.

Additional investigation may be required to further characterize or determine the identity of the plasmid.
3) If none of the bands at approximately 460, 349, 320, or 268 bp is present, the sample is unlikely to contain the pXO1 plasmid and should be characterized as pXO1⁻.
   b. pXO2
      1) If all of the bands at approximately 250, 228, and 207 bp are present, the sample contains the pXO2 plasmid or a plasmid with DNA sequence homology in the regions of the particular targets and should be characterized as pXO2⁺.
      2) If some (but not all) of the bands at approximately 250, 228, or 207 bp are present, the species of the sample should be considered.
   a) If the sample is DNA from B. anthracis, it is likely that it contains the pXO2 plasmid, possibly with a mutation/deletion for one or more of the targets, and should be characterized as pXO2⁺.
   b) If the sample is not DNA from B. anthracis and not from a strain known to harbor a homologous plasmid, the strain may contain a plasmid with DNA sequence homology to pXO2.
   Additional investigation may be required to further characterize or determine the identity of the plasmid.
      3) If none of the bands at approximately 250, 228, or 207 bp is present, the sample is unlikely to contain the pXO2 plasmid and should be characterized as pXO2⁻.

### TROUBLESHOOTING

A Primer dimers: Primer dimers, including some large ones (˜75 bp), are occasionally seen on the gel. In the products of this multiplex PCR, faint or fuzzy bands smaller than 100 bp can be disregarded; they have no bearing on the results, in particular embodiments.

B. Little or no PCR product: Poor quality of PCR templates, primers or reagents may lead to PCR failures. Using appropriate PCR controls in the assay will help eliminate or verify this as a possibility.

C. Little or no band in gel: Make sure the appropriate percentage of gel was used. In at least certain embodiments, the wrong percentage of gel can cause no band to appear, or even a slight faint band to appear when it should be bolder.

TABLE 1
Primer Sequences and Expected Amplicon Sizes

| | | Final Conc. in | Approx. |
|---|---|---|---|
| Primer | | Primer Mix | Amplicon |
| Name | Primer Sequence (5′→3′) | (μM) | Size |
| E517F | gcc agc agc cgc ggt aa | 1 | 555 bp |
| E1072R | cga gct gac gac arc cat gca | 1 | |
| lefF | cta tca aca ctg gag cga ttc ttt atc tg | 2 | 460 bp |
| lefR | ggt act tcc aat gga ttg atg taa taa agc | 2 | |
| ORF53 F | aca aca gcg ctt ttt cta acg ctt t | 0.4 | 349 bp |
| ORF53 R | tgt tag ccc ata ttg gtg ctt tca c | 0.4 | |
| pagA F | ggc att taa tct tgc tgt atc agc g | 0.15 | 320 bp |
| pagA R | tgg cag ctt atc cga ttg tac atg t | 0.15 | |
| cya F | tgc ccc cga cat gtt tga gt | 0.25 | 268 bp |
| cya R | att caa tcc ctt tgt agc cac acc | 0.25 | |
| capA2 F | cag gag cta ttg caa cga aag aac aac cag | 0.25 | 250 bp |
| capA2 R | aca tca aaa gat tga agt aca tgc gga tgg | 0.25 | |
| ORF7 F | cgg gcg aaa ata aaa aag aag gta a | 0.7 | 228 bp |
| ORF7 R | ttg ccg cct tat tta cat gtg att t | 0.7 | |
| capB F | tcc gga tcc agg agc aat ga | 0.25 | |
| capB R | tcc cta gca aac tgc tca gta cga t | 0.25 | 207 bp |
| sspE F | gag aaa gat gag taa aaa aca aca a | 0.5 | 188 bp |
| sspE R | cat ttg tgc ttt gaa tgc tag | 0.5 | |

TABLE 2
Exemplary Multiplex PCR Formulation

| | Stock | | Concentration | | Volume per Single |
|---|---|---|---|---|---|
| Reagent | Concentration | | in Mastermix | | Reaction |
| 10X PCR Buffer | 10X | | 1X | | 5 μL |
| dNTPs | 2.5 | mM | 350 | μM | 7 μL |
| MgCl₂ | 50 | mM | 7.5 | mM | 7.5 μL |
| Primer Mix (from | n/a | | n/a | | 21.5 μL |
| Table 1) | | | | | |
| Platinum Taq | 5 | U/μL | 3 | U | 0.6 μL |

| | | | |
|---|---|---|---|
| H₂O | n/a | n/a | 4.4 μL |

| | |
|---|---|
| Mastermix Total per Reaction: | 46 μL |

Table 2 shows the formulation of a single reaction (testing of a single sample). The volumes should be multiplied in order to produce enough mastermix to perform the assay on the desired number of samples.

TABLE 3
Primer Mix Formulation Volumes

| | | Final | Volume for | |
|---|---|---|---|---|
| | Working Stock | Concentration | Single | Volume for |
| | Concentration | in Primer Mix | Reaction | 10 Reactions |
| Primer | (μM) | (μM) | (μL) | (μL) |
| E517F | 20 | 1 | 2.5 | 25 |
| E1072R | 20 | 1 | 2.5 | 25 |
| lef F | 50 | 2 | 2 | 20 |
| lef R | 50 | 2 | 2 | 20 |
| ORF53 F | 20 | 0.4 | 1 | 10 |
| ORF53 R | 20 | 0.4 | 1 | 10 |
| pagA F | 20 | 0.15 | 0.375 | 3.75 |
| pagA R | 20 | 0.15 | 0.375 | 3.75 |
| cya F | 20 | 0.25 | 0.625 | 6.25 |
| cya R | 20 | 0.25 | 0.625 | 6.25 |
| capA2 F | 20 | 0.25 | 0.625 | 6.25 |
| capA2 R | 20 | 0.25 | 0.625 | 6.25 |
| ORF7 F | 20 | 0.7 | 1.75 | 17.5 |
| ORF7 R | 20 | 0.7 | 1.75 | 17.5 |
| capB F | 20 | 0.25 | 0.625 | 6.25 |
| capB R | 20 | 0.25 | 0.625 | 6.25 |
| sspE F | 20 | 0.5 | 1.25 | 12.5 |
| sspE R | 20 | 0.5 | 1.25 | 12.5 |
| | | | Total | 215 |
| | | | Volume: | |

### Example 4

### Exemplary Embodiments

Embodiments of the invention include a multiplexed combination of primers that target a chromosomal mutation specific to Bacillus anthracis in order to identify B. anthracis at the species level and detect and characterize the virulence plasmids. The invention is useful at least to determine whether or not a B. anthracis sample is a Select Agent. Such determination is helpful because, for example, when shipping materials one must ensure that the proper regulatory requirements are met depending on whether the material is a Select Agent or not.

The multiplexed combination of primers target the following:
a) sspE repeat, a chromosomal mutation specific to B. anthracis, allowing species level identification; and
b) Four multiply redundant targets (lef, pag, and cya and separate target located distant from pXO1 pathogenicity island, ORF53). The separate target, ORF53 serves to provide further verification of the presence of pXO1 in the event any of the other targets have been changed, modified, or are not present; and
c) Three multiply redundant targets (two capsule genes, capA and capB and a separate target located a distance from pXO2, ORF7). The separate target, ORF7 serves to provide further verification of the presence of pXO2 in the event any of the other targets have changed, modified, or are not present; and
d) 16S ribosomal RNA (rRNA), serving as an internal control.

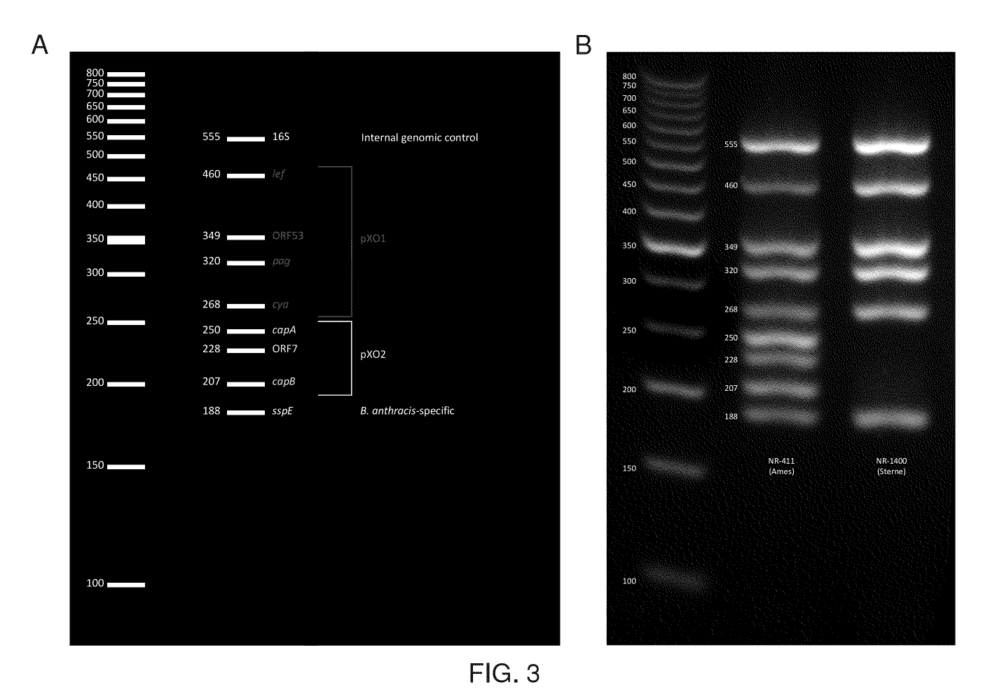

FIG. 3 panel A shows a graphic representation of the band pattern from a pXO1⁺/pXO2⁺B. anthracis sample; all bands (i.e., amplicons from 16S rRNA, 4 targets on pXO1, 3 targets on pXO2, and sspE) are present. A commercial DNA ladder is shown at left for size comparison. FIG. 3 panel B shows actual gel electrophoresis results from the multiplex PCR on two samples, NR-411 (pXO1⁺/pXO2⁺) and NR-1400 (pXO1⁺/pXO2⁻); the results for NR-1400 show no amplicons from the 3 targets located on pXO2, indicating the plasmid is most likely not present.

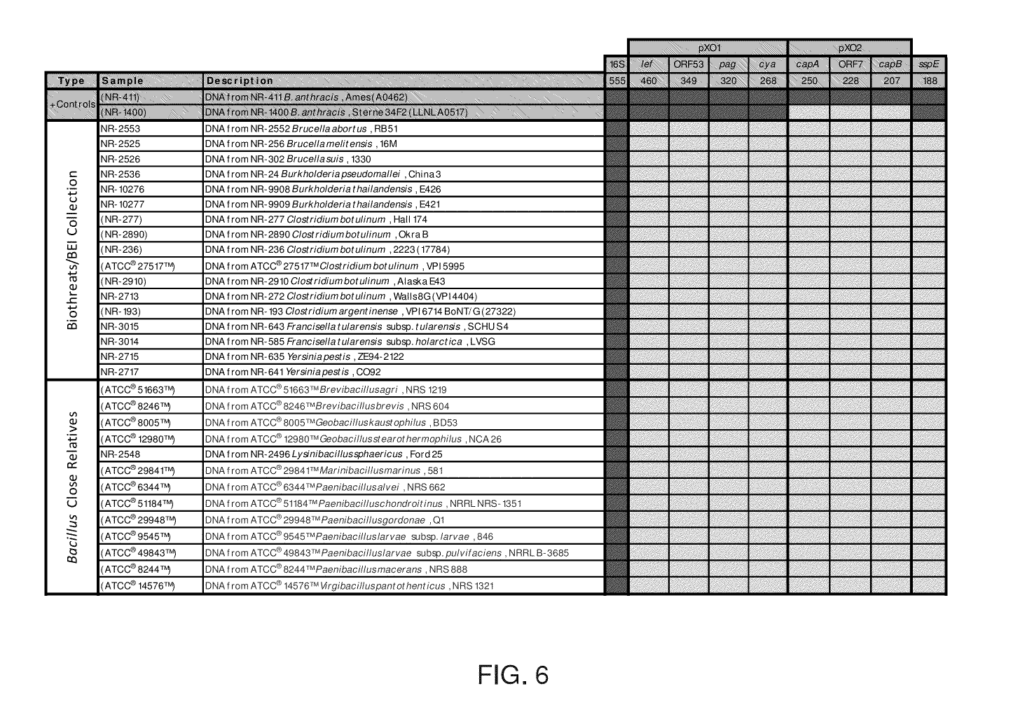

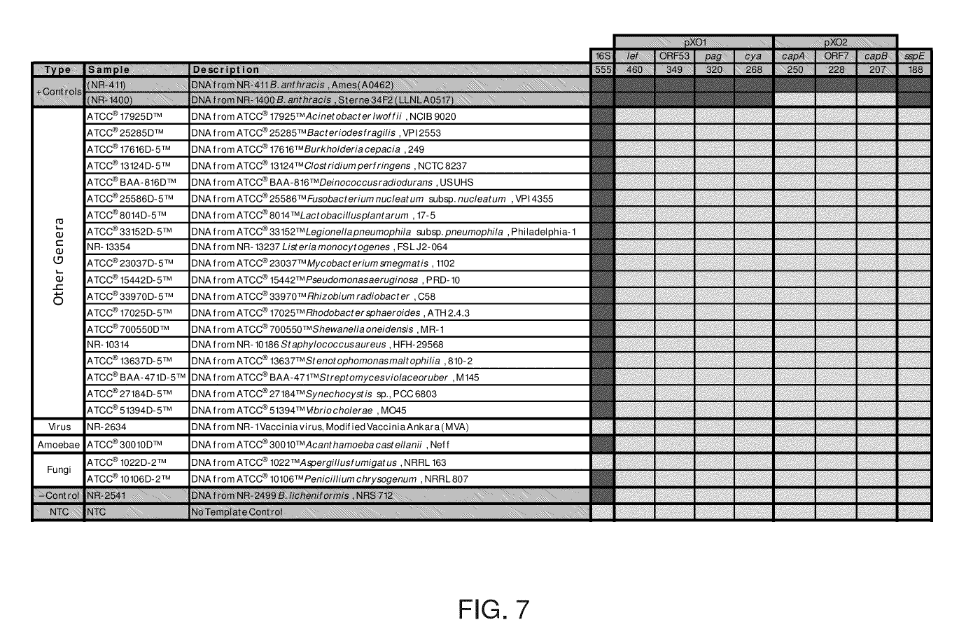

FIGS. 4-7 show results of inclusivity and exclusivity testing. Samples of B. anthracis as well as from other genera and kingdoms are listed on the leftmost column of the figures. The results are coded: dark gray indicates that an amplicon for the particular target was observed via gel electrophoresis, while light gray indicates that no amplicon was observed for a particular target. In FIG. 5, for NR-4195, there is a novel plasmid partially homologous to pXO1; for NR-10050, pBCXO1≈99.6% identical to pXO1; for ATCC® 10987D-5™, pBC10987≈40% identical to pXO1; for (NR-610), there is a novel plasmid partially homologous to pXO2; and for (ATCC® 35866™), pAW63≈53% identical to pXO2. In FIG. 7, for ATCC® 30010D™, there is rRNA homologous to bacterial 16S and for ATCC® 10106D-2™, there is 18S homologous to bacterial 16S.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
Kim, K., et al. “Rapid genotypic detection of Bacillus anthracis and the Bacillus cereus group by multiplex real-time PCR melting curve analysis.” FEMS Immunol Med Microbiol 43 (2), 301 (2005).
Okinaka, R. T., et al. “Sequence and Organization of pXO1, the Large Bacillus anthracis Plasmid Harboring the Anthrax Toxin Genes.” J. Bacteriol. 1999, vol. 181(2); p. 6509-6514
Sozhamannan, S., et al. “The Bacillus anthracis chromosome contains four conserved, excision-proficient, putative prophages.” BMC Microbiology 2006, 6:34.
Van der Auwera, G. A. et al. “Conjugative plasmid pAW63 brings new insights into the genesis of the Bacillus anthracis virulence plasmid pXO2 and of the Bacillus thuringiensis plasmid pBT9727.” BMC Genomics 2005, 6:103.
Wang, Y., and Qian, P-Y. “Conservative Fragments in Bacterial 16S rRNA Genes and Primer Design for 16S Ribosomal DNA Amplicons in Metagenomic Studies.” PLoS One 4(10):e7401 (2009).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of testing for the presence or absence of Bacillus anthracis in a sample, comprising the steps of assaying for the presence of the following targets in nucleic acid from the sample:
a species-specific target;
two or more targets on pXO1 plasmid, wherein a first target is a virulence gene and a second target is distant on the plasmid from the first target and/or is a non-virulence gene;
two or more targets on pXO2 plasmid, wherein a first target is a virulence gene and a second target is distant on the plasmid from the first target and/or is a non-virulence gene; and
optionally a bacterial genomic positive control target.

2. The method of claim 1, further defined as assaying for two or three virulence gene targets on pXO1 plasmid.

3. The method of claim 1, further defined as assaying for two or three virulence gene targets on pXO2 plasmid.

4. The method of claim 1, wherein the species-specific target comprises sspE or is one or more B. anthracis-specific prophage(s).

5. The method of claim 4, wherein the species-specific target comprises a mutation in sspE.

6. The method of claim 1, wherein the first target on the pXO1 plasmid is selected from the group consisting of lef, pag, cya, and combinations thereof.

7. The method of claim 1, wherein the first target on the pXO2 plasmid is selected from the group consisting of capA, capB, capC, capD, capE, and combinations thereof.

8. The method of claim 1, wherein the bacterial genomic positive control target is a housekeeping gene.

9. The method of claim 8, wherein the housekeeping gene comprises ribosomal RNA.

10. The method of claim 9, wherein the ribosomal RNA is 16S RNA.

11. The method of claim 1, wherein the second target on the pXO1 plasmid is ORF53.

12. The method of claim 1, wherein the second target on the pXO2 plasmid is ORF7.

13. The method of claim 1, further comprising the step of obtaining the sample.

14. The method of claim 1, wherein the sample is suspected of comprising B. anthracis or known to comprise B. anthracis.

15. The method of claim 1, wherein the sample is or is from a powder, liquid, gel, aerosol, solid, or mixture thereof.

16. The method of claim 1, wherein the sample is from or is an unknown substance.

17. The method of claim 1, wherein the sample is from a repository.

18. The method of claim 1, wherein the sample is to be deposited in a repository.

19. The method of claim 1, further comprising the step of transporting the sample.

20. The method of claim 1, wherein the nucleic acid is purified nucleic acid.

21. The method of claim 1, wherein the assaying comprises amplification of one or more of the targets.

22. The method of claim 21, wherein the amplification comprises polymerase chain reaction.

23. The method of claim 1, comprising the steps of assaying for the presence of the following targets in nucleic acid from the sample:
sspE;
lef, pag, and cya;
ORF53;
ORF7;
capA and capB; and
16S RNA.

24. A kit comprising primers suitable for amplification of the following targets:
a species-specific target;
one or more targets on pXO1 plasmid, wherein a first target is a virulence gene and a second target is distant on the plasmid from the first target and/or is a non-virulence gene;
one or more targets on pXO2 plasmid, wherein a first target is a virulence gene and a second target is distant on the plasmid from the first target and/or is a non-virulence gene; and
optionally a bacterial genomic positive control target.
